(19) United States
(12) Patent Application Publication
(10) Pub. No.: US 2022/0369945 A1
(43) Pub. Date: Nov. 24, 2022

(54) IOT ENABLED PORTABLE MULTIPLE STIMULI REINFORCED NON-NUTIRIVE FEEDING TRAINING SYSTEM

(71) Applicant: Sarvesh Santosh Karkhanis, Kasarvadavali, Thane (IN)

(72) Inventor: Sarvesh Santosh Karkhanis, Kasarvadavali, Thane (IN)

(21) Appl. No.: 17/772,448

(22) PCT Filed: Nov. 6, 2020
(86) PCT No.: PCT/IB2020/060484

§ 371 Date: Apr. 27, 2022

(30) Foreign Application Priority Data
Oct. 29, 2019 (IN) 201921043719

(51) Int. Cl. A61B 5/03; A61B 5/00; A61J 17/00

(52) U.S. Cl. CPC A61B 5/038; A61B 5/682; A61J 17/10

(57) ABSTRACT

Disclosed is an IoT enabled, portable, multiple stimuli reinforced non-nutritive Sucking Training System, Method and Device that implements a function that measures suction pressure of a non-nutritive sucking event produced by a pre-mature infant; then converts the measured pressure value using transducer to digital form; then transmits the pressure value in digital form using IoT (Internet-of-Things) controller to a server and/or to an app and/or a software installed on a smartphone device. The app and/or software installed on the smartphone device which receives the pressure measurement uses an algorithm to produce a stimulus such as music and/or haptic feedback for the pre-mature infant, to train the premature infant to suck optimally; thereby improving self-feeding capability of the premature infant.

### BACKGROUND OF THE INVENTION

Pre-mature infants have a sub-optimal control on their mouth muscles, which prevents them to feed themselves properly. Due to insufficient feeding, serious issues arise such as malnutrition, that can result in permanent disabilities or even deaths of premature infants. An established solution to this daunting issue, is to train the pre-mature infants using various methods to control their mouth muscles, so that they can feed optimally and gain enough nutrition. It has been shown that these pre-mature infants can be trained to control their mouth muscles using different stimuli such as music, which is synced with their non-nutritive sucking responses.

Currently there exists only a handful of prior art that describe devices which measure non-nutritive sucking pressure of an infant. One such prior art U.S. Pat. No. 5,830,235 uses a combination of physically large consumer appliances such as cassette player and physically large instruments to measure the non-nutritive sucking pressure using a transducer and play music as a stimulus. Another prior art U.S. Pat. No. 4,554,919 describes a non-therapeutic so-called amusement device for infants that consist of a pacifier. The prior art Australian Patent 2014207317 describes a handheld device used to assess the non-nutritive sucking events.

None of the foregoing devices and designs are believed to implement an IoT (Internet-of-Things) based system connected to internet which provides real-time data of non-nutritive sucking events to healthcare practitioners; and provide stimuli such as music and/or recorded voice and/or haptic feedback to the user (premature infants) at the same time.

The invention presented here satisfies the foregoing needs by implementing an IoT (Internet-of-Things) based system which comprises of a trainer device for user; a computer server to store and parse data; and a smartphone app to view data for healthcare practitioners; app which also plays a desired stimuli.

In the presented invention, an IoT (Internet-Of-Things) and/or Bluetooth communication hardware and software is implemented so that such devices can communicate with server and/or smartphone devices and/or other digital devices which are also connected via internet and/or Bluetooth.

More preferably, in the presented invention, a probe made of medical-grade silicone is used, which is connected to a pressure transducer. When an infant sucks onto the probe, the sucking pressure is measured and converted to digital form using the pressure transducer. The pressure measurement in digital form is fetched to an IoT (Internet-of-things) and/or Bluetooth module. The IoT (Internet-of-Things) and/or Bluetooth module transmits the pressure in digital form to a server and/or smartphone connected to the internet. An app or software installed on the smartphone device on the receiving end reads the pressure value. If the pressure value is higher than a specific healthcare practitioner set or default value in the app, the smartphone would play a music and/or activate a haptic feedback. In this way the music and/or haptic feedback is synced with non-nutritive sucking pressure values.

More preferably, in the presented invention, the app can play any type of music, instead of playing only a set loop. User's preference of music can be determined by healthcare practitioners through simple experimentation. From a provided set of music loops, the music which the infant being treated most responds to, is played using smartphone device. The system can also implement a recording capability, thereby enabling the parents, particularly the mother of the user to record her voice. This recorded voice of mother and/or parents can also be played as a stimulus response, instead of a music loop.

Hence in summary, having sub-optimal control on mouth muscles is a major issue for premature infants which prevents them to have optimum nutrition; and can result is permanent disabilities or even deaths. The invention presented here is a solution to the daunting issue. This invention creates an IoT enabled system to train pre-mature infants to feed themselves, thereby contributing in saving lives of premature infants.

### BRIEF DESCRIPTION OF THE DRAWINGS

Having thus briefly described the invention, the same will become better understood from the following detailed discussion, taken in conjunction with the appended drawings wherein

FIG. 1 Is a Flowchart that explains the implemented system in accordance with the invention.

FIG. 2 Is a schematic block diagram of the first embodiment of the System in accordance with the invention

FIG. 3 Is drawing of the first embodiment of the physical device used as part of the System in accordance with the invention. This device is referred to as the Trainer Device.

FIG. 4 Is a drawing of the first embodiment of the electronic circuit that depicts various components of the Trainer Device, which is one of the parts of the implemented System in accordance with the invention.

FIG. 5 Is a drawing depicting a screen in the smartphone app, which visualizes pressure sensor readings; where the app is one of the parts of the implemented system in accordance with the invention

FIG. 6 and FIG. 7 are drawings depicting screens in the smartphone app, which contains some controls to be used by a healthcare practitioner who is treating the User (Premature infant); where the app is one of the parts of the implemented System in accordance with the invention.

### DETAILED DESCRIPTION OF THE DRAWINGS

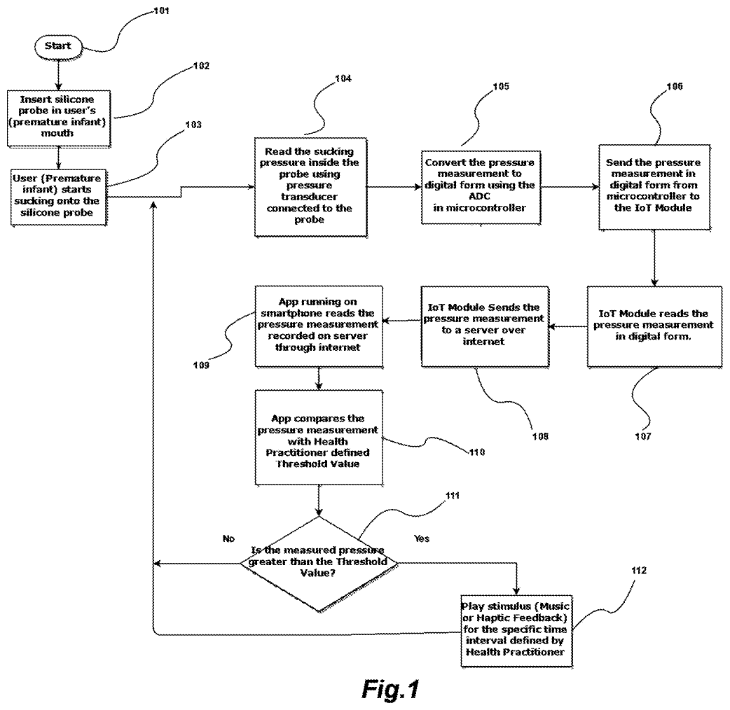

FIG. 1 is a Flowchart that shows the method and process that takes place throughout the system. Initially 101 The silicone probe is inserted 102 in to the user's (pre-mature infant) mouth. The premature infant then starts sucking 103 onto the probe. The sucking pressure is read by the pressure transducer 104 connected to the probe. The pressure is then converted to a digital form 105 and is sent 106 to an IoT (Internet-of-Things) module. The IoT (Internet-of-Things) module reads the pressure measurement in digital form 107 and sends it to a server 108 over the internet. The foregoing App running on a smartphone, reads 109 the pressure measurement recorded on the server, through internet. The App then compares the real-time pressure measurement 110 with a health practitioner defined threshold value. If the measured pressure is greater than the threshold value 111, then the App plays a stimulus 112 such as music, recorded voice, haptic feedback or a combination of any of them. The infant is encouraged to play more stimulus by sucking onto the probe more, thereby training the infant to suck onto the probe.

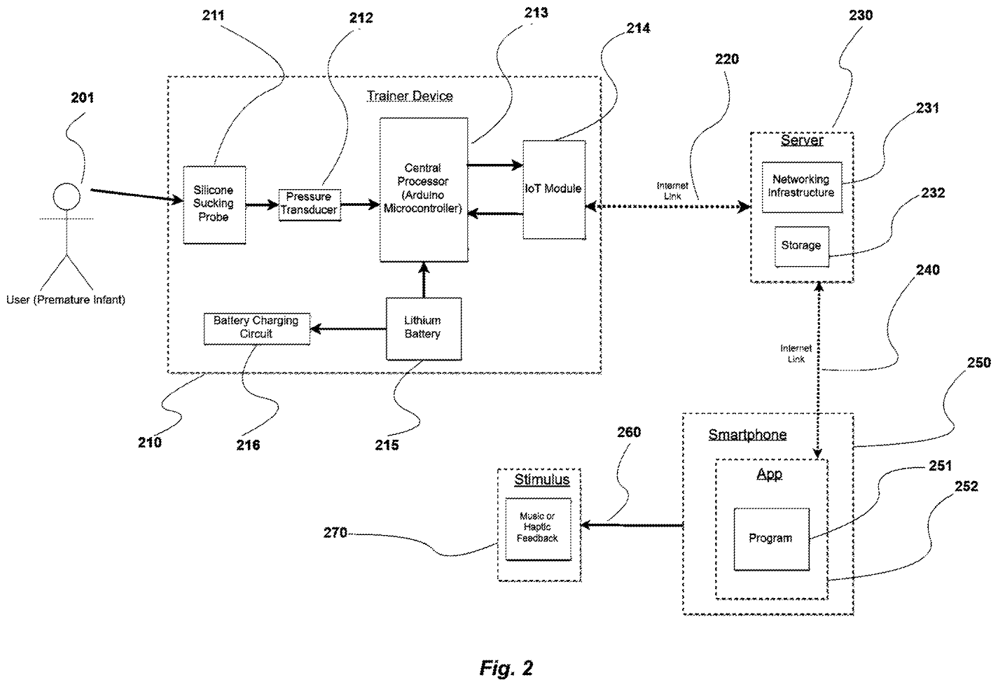

FIG. 2 contains the Block Diagram of first embodiment of the system, that depicts all the physical as well as logical components of the of the implemented system in accordance with the invention. In brief, in the system, there is the user (Pre-mature infant) 201 who uses the Trainer Device 210 which communicates with the server 230 over internet which turn communicates with a smartphone 250; which plays a stimulus 270. The user 201 who is intended to be a premature infant, sucks onto the silicone probe 211 creating a negative pressure inside the probe 211. The pressure transducer 212 which is connected to the silicone probe 211, then reads the sucking pressure (negative pressure) inside the probe 211, and sends the analog pressure value to the Central Processor 213. The Central Processor 213 then converts the pressure value to a digital form using ADC (Analog to Digital Converter). In the next step, the Central Processor 213 communicates with an IoT module 214. Now the IoT module 214 receives pressure measurement in digital form from the microcontroller 213. In the next step, the IoT module 214 communicates with a server 230 through internet link 220, and sends the pressure measurement to the server 230. The server 230 is always connected to the internet through its network infrastructure 231. The server 230 retains the received pressure measurement in its storage 232. In the next step, the app 252 installed on a smartphone device 250 communicates with the server 230 via an internet link 240 and retrieves the pressure measurement from the server 230. In the next step, the program logic 251 in the app 252 determines whether the real-time sucking pressure obtained from the server 230 is greater than a healthcare practitioner-set threshold pressure. If the sucking pressure is higher than the threshold pressure, then the app 252 running inside smartphone device 250 communicates 260 with its peripherals and plays a stimulus 270 such as music or haptic feedback for a healthcare practitioner specified amount of time. Finally, this stimulus 270 synced with the sucking action is used to train the pre-mature infant 201 to feed itself. The described loop is continuously followed by the system as long as it is powered. The model of pressure transducer 212 used in the first embodiment of the Trainer Device was MPX5050DP manufactured by NXP Semiconductor. The model of central processor 213 used in the first embodiment of Trainer Device was Arduino Uno microcontroller which implements a ATMega328 16 Mhz, 8-bit chip manufactured by Atmel. In the first embodiment of the Trainer Device, the silicone probe 211 was connected to the Vacuum port of the MPX5050DP pressure transducer 212 using an air-tight silicone tubing. The microcontroller is powered by a lithium-ion battery 215. The microcontroller board 213 provides power to other peripherals. The lithium battery 215 is charged using a charging circuit 216. The model of IoT module 214 used in the first embodiment of the system was ESP-32 manufactured by Espressif Systems. The model of the smartphone 250 used in first embodiment of the system was Nexus 5 manufactured by LG Electronics, running Android operating system; and the inventor-created app 252 was running on the Android system.

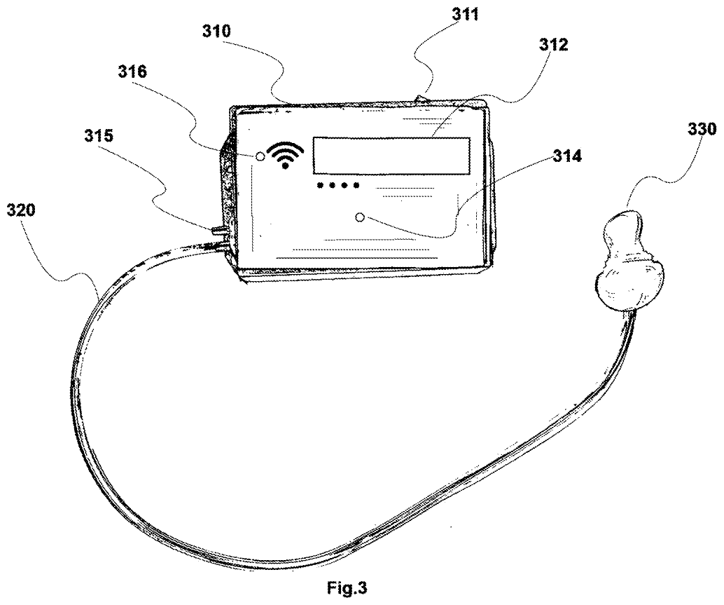

FIG. 3 contains physical drawing of the Trainer Device 210 described in FIG. 2. The Trainer Device 210 is one of the parts of the described System in accordance with the invention. The Trainer Device contains an enclosure 310 made out of ABS plastic. The dimensions of the enclosure 310 are 120 mm×80 mm×40 mm. The enclosure 310 encloses a Lithium-Ion Battery and has an on/off switch 311 to witch on or off the Trainer Device. The enclosure 310 has an LCD screen 312 and few buttons below the LCD screen for showing different user settings. There is a power status LED 314 on the enclosure 310 which glows when the Trainer Device is powered. There is also a WiFi status LED 316 which shows the WiFi connection status of the enclosed IoT (Internet-of-Things) module. The inlet ports 315 of the enclosed pressure transducer are on a side of the enclosure. The silicone sucking probe 330 is connected to the vacuum pressure port 315 of the transducer via a silicone tube 320.

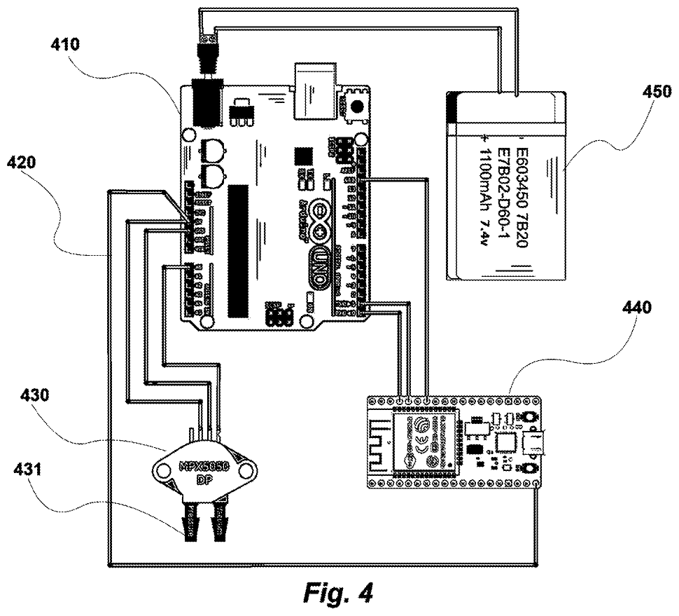

FIG. 4 contains physical drawing of the electronic components and circuitry enclosed inside the enclosure 310 described in FIG. 3. In the first embodiment of the System, the enclosure 310 which is part of the Trainer Device 210, contains an Arduino Uno microcontroller 410 as the central processor. An MPX5050DP pressure transducer 430 which has two pressure ports 431, is connected to the Arduino Uno microcontroller 410 using electrical conducting wires 420. An ESP-32 IoT (Internet-of-Things) module 440 is connected via serial port to the Arduino Uno microcontroller 410. A Lithium-Ion battery 450 is connected to the Arduino Uno microcontroller 410 powering the microcontroller. The Arduino Uno microcontroller 410 provides power to other peripherals such as the pressure transducer 430 and the IoT (Internet-of-Things) module 440.

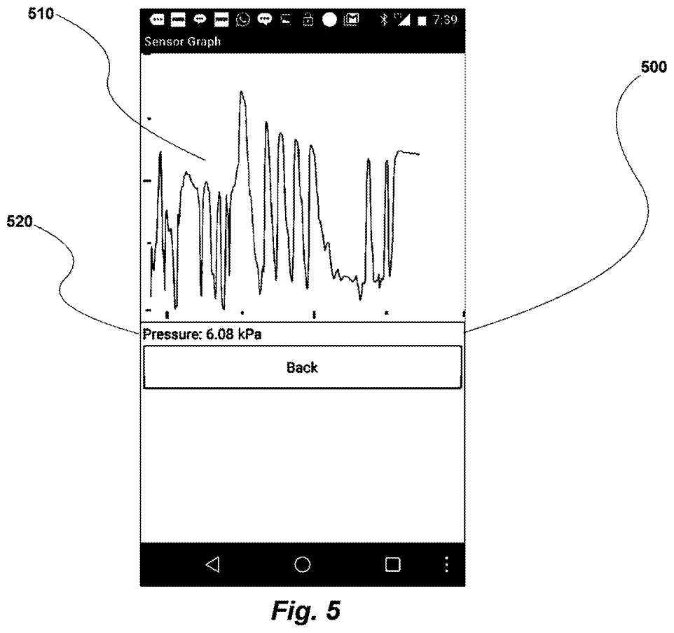

FIG. 5 depicts drawing of a screen 500 from the app 252 running on the smartphone 250. The App 252 has an ability to display a continuous real-time graph 510 of the pressure measurements read by the pressure transducer 212 from inside of the silicone probe 210. The App UI 500 also displays the real time pressure measurement 520, updated every 10th of a second.

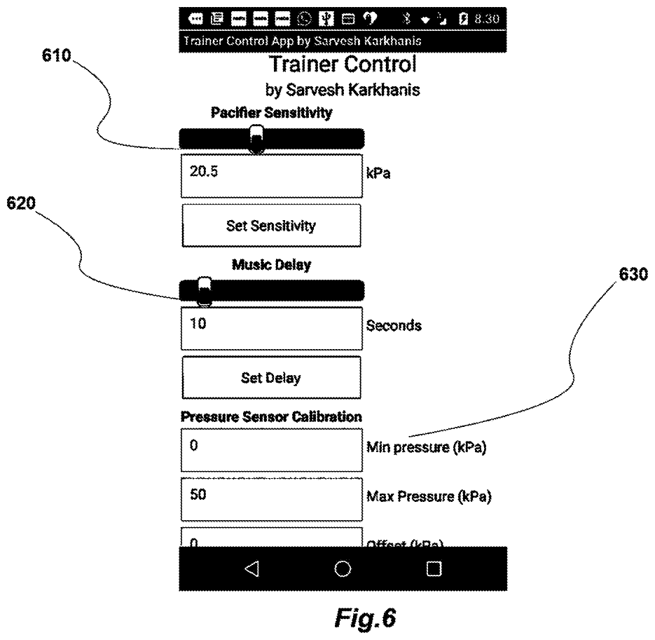

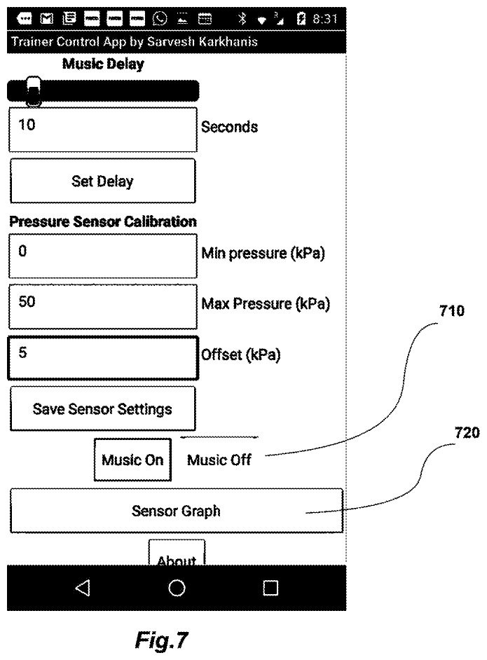

FIG. 6 and FIG. 7 shows some other functions from a screen of the smartphone App 252 created as part of this invention. The pressure measurements can be calibrated through software using the provided fields 630 in the App. The Pacifier sensitivity 610 can be set, and the delay between repetitive cycles of the stimulus can be adjusted using provided 620 options. The stimulus such as music or haptic feedback can be turned on or off 710. All these options are supposed to be used by the health practitioner who is treating the User 201 (premature infant). The graph of real-time pressure values over time 510 can also be viewed by the health practitioner anytime while using the provided feature 720.

### REFERENCES CITED

### US Patent Documents

1) U.S. Pat. No. 5,830,235—November 1998—Standley et al.
2) U.S. Pat. No. 4,554,919A—December 1984—Hubert
3) AU2014207317—April 18—Aron et al.

### Other Publications

1) A. J. DeCasper & W. P. Fifer, “Of Human Bonding: Newborns Prefer Their Mothers'Voices”, 1980, pp. 1174-1176
2) Standley, J. M., “The effect of music-reinforced nonnutritive sucking on feeding rate of premature infants”, 2003, Journal of Pediatric Nursing, 18(3), 169-173. doi:10.1053/jpdn.2003.34
3) Chorna, O. D., Slaughter, J. C., Wang, L., Stark, A. R., & Maitre, N. L., “A Pacifier-Activated Music Player With Mothers Voice Improves Oral Feeding in Preterm Infants”, 2014, Pediatrics, 133(3), 462-468. doi:10.1542/peds.2013-2547
4) Cevasco, A. M., & Grant, R. E., “Effects of the Pacifier Activated Lullaby on Weight Gain of Premature Infants”, 2005, Journal of Music Therapy, 42(2), 123-139. doi:10.1093/jmt/42.2.123
5) Cheour-Luhtanen, M., Alho, K., Sainio, K., Rinne, T., Reinikainen, K., Pohjavuori, M., Renlund, M., Altonen, O., Eerola, O., Näätänen, R., “The ontogenetically earliest discriminative response of the human brain”, 1996, Psychophysiology, 33(4), 478-481. doi:10.1111/j.1469-8989.1996.tb01074.x

What is claimed is:

1. A system which contains a portable device, a server and a smartphone app; in which the portable device is for measuring non-nutritive sucking pressure of a non-nutritive sucking event from an infant, comprising of a probe to suck on; a transducer connected to the probe; an IoT (Internet-of-Things) and/or Bluetooth module connected to the transduce via a microcontroller; and a server and/or a smartphone device to collect the measured pressure data; and a smartphone to play music and/or recorded voice and/or provide haptic feedback.

2. The portable device in claim 1; wherein the portable device has a weight less than 1.5 Kilograms

3. The portable device in claim 1; wherein the portable device contains a lithium battery

4. The probe in claim 1; wherein the probe is made of silicone, has a shape similar to human female nipple and is connected to the transducer inside the portable device using an air-tight silicone tubing.

5. The portable device in claim 1; wherein the transducer it contains converts pressure inside the probe in claim 4 to analog form, which in turn is converted by a microcontroller to digital form, meaning in a machine readable binary form.

6. The portable device in claim 1; wherein the IoT (Internet-of-Things) module reads the pressure measurement in digital form and transmits it over internet to a specified server and/or a smartphone device using any of the internet and IoT (internet-of-things) protocols including but not limited to TCP/IP, UDP, SSL and MQTT.

7. The portable device in claim 1; wherein the Bluetooth module reads and transmits pressure measurement in digital form over Bluetooth frequency.

8. The portable device in claim 1; wherein a server means any computing resource connected to the internet.

9. The portable device in claim 1; wherein a smartphone device means any handheld computing device with cellular communication capability.

10. The portable device in claim 1; wherein the transducer has measuring range anything between negative 100 kilo pascal pressure to 100 kilo pascal pressure.

11. The portable device in claim 1; wherein the transducer measures pressure relative to the relative ambient pressure.
